(19) United States
(12) Patent Application Publication
(10) Pub. No.: US 2015/0140523 A1
(43) Pub. Date: May 21, 2015

(54) Computer Implemented System and Method for Determining, Managing and Optimizing Calorie Intake of a User

(71) Applicant: Mohan DEWAN, Maharashtra (IN)

(72) Inventor: Mohan Dewan, Pune (IN)

(21) Appl. No.: 14/360,141

(22) PCT Filed: Nov. 5, 2012
(86) PCT No.: PCT/IN2012/000723

§ 371 Date: May 22, 2014

(30) Foreign Application Priority Data
Nov. 23, 2011 (IN) 3290/MUM/2011

(51) Int. Cl. G09B 19/00; A61B 5/00; A61B 5/11

(52) U.S. Cl. CPC G09B 19/0092; A61B 5/1118; A61B 5/7246
USPC 434/127

(57) ABSTRACT

A system and method for determining, managing and optimizing calorie intake of a user have been disclosed. The system determines the calorie intake of a user based on basal metabolic rate and body mass index of the user and accordingly generates a customized menu for the user. The customized menu generated for the user includes food items having appropriate calories which help the user in maintaining good health. The system also enables the user to specify a calorie requirement and subsequently generates a food menu comprising food items having a calorie value specified by the user.

### FIELD OF THE DISCLOSURE

The present disclosure relates to the field of food management. More particularly the present disclosure relates to the field of managing and optimizing calorie intake. Still more particularly the present disclosure relates to a computer implemented system that enables user sto track, mage and optimize their calorie intake.

### BACKGROUND

A healthy life is governed by balanced diet, adequate exercise to the body and a healthier approach towards life. A balanced diet ensures that essential nutrients are supplied in adequate quantity to the body. Other than the nutrients it is the amount of calories in the diet that helps maintain good health and a good personality.

However, people often find it difficult to balance the intake of calories and end up consuming food that has either higher calories or lower calories. Apart from having food stuffs that have high calories, people often fail to burn the excess amount of calories present in their body, thereby putting on weight. People who eat food low on calories tend to lose weight rapidly thereby having a lanky personality. It is notable that the imbalance between the intake of calories and expenditure of calories leads to a plurality of health related issues apart from rapid weight gain/loss. The imbalance between the intake and expenditure of calories affects a plurality of factors including life style and personal appearance.

Selection of a diet plan or eating habits is often governed by personal tastes and choices. Intake of food rich in terms of calories and failure to effectively utilize the excessive calories results in weight gain. A diet that provides excess calories often stimulates weight gain as the excessive, un burnt calories get deposited in the body as fat. Presence of excessive fat in the body is termed ‘obesity’. Research studies have confirmed that obesity is one of the causes for high blood sugar level, coronary heart disease, high blood pressure, kidney failure, reduced body metabolic rate and back ache. On the other hand rapid reduction in body weight, which is also influenced by the calorie intake and burning of calories, results in low blood sugar level, breakdown of body tissues and low blood pressure.

Dieticians advise people to maintain good health by consuming food that gives them optimal amount of calories. Optimization of intake of calories includes having a diet with right proportion of carbohydrates, fats and proteins. Further, adapting physical activities such as cycling, swimming, jogging, brisk walking, dancing, yoga and skipping help in burning the excess calories thereby helping people maintain a balance between the calorie intake and calorie expenditure.

Tracking the intake of calories of a user includes tracking at least the body mass index (BMI) and basal metabolic rate (BMR). Basal metabolic rate is the number of calories a person burns in a given day if he/she is totally at rest. Body mass index provides a gross estimation of the amount of fat in the human body. The numerical value of body mass index in the range 24-25 indicates a healthy body weight. If the body mass index is below 24, it indicates underweight and if the body mass index is beyond 25, it indicates a gain in the bodyweight. Calorie counting, as the name suggests includes tracking the balance between intake of calories and expenditure of calories.

Further, there has been an increase in the trend of eating out. People tend to consume food in outside eateries such as fast food joints, snack centers, restaurants and hotels. However, people eating out have no way of determining the calorie content present in the food served in the eateries. Moreover, the menu provided in outside eateries does not contain any information about the nutritional contents/nutritional values present in the food items. The menu provided in outside eateries cannot be customized as per an individual's needs and the individual is forced to select whatever is available for serving. Moreover, most of the people are unaware of the nature of diet to be followed for optimizing the intake of calories and nutrients. Therefore, there was felt a need for a system which:
keeps track of the calorie intake of a user;
generates a food menu (menu card) customized according to users' individual needs;
generates a food menu based on the calorie requirement specified by individual users; and
assists a user in maintaining good health by recommending him/her a balanced diet.

### OBJECTS

Some of the objects of the present disclosure are described herein below:

It is an object of the present disclosure to ameliorate one or more problems of the prior art or to at least provide a useful alternative.

An object of the present disclosure is to provide a system that keeps track of the calorie intake of a user.

Yet another object of the present disclosure is to provide a system that generates a food menu (menu card) customized according to users' individual needs.

Still a further object of the present disclosure is to provide a system that generates a food menu based on the calorie requirement specified by individual users.

Another object of the present disclosure is to provide a system that assists a user in maintain good health by recommending the user a balanced diet.

Yet another object of the present disclosure is to provide a system that assists users in avoiding health risks.

Still a further object of the present disclosure is to provide a system that assists users in maintain/reducing their respective body weight.

Other objects and advantages of the present invention will be more apparent from the following description when read in conjunction with the accompanying figures, which are not intended to limit the scope of the present invention.

### SUMMARY

The present disclosure envisages a computer implemented system for determining, managing and optimizing calorie intake of a user. The system, in accordance with the present disclosure includes:
a user interface configured to enable the user to generate a request for a food menu, the user interface further configured to receive from the user values corresponding to a plurality of physiological factors corresponding to the user;
a deriving module having access to the values corresponding to a plurality of physiological factors associated with individuals, the deriving module configured to derive standard values for body mass index and basal metabolic rate depending upon the values of the physiological factors;
a metabolic database configured to store values of the physiological factors and the corresponding standard values for body mass index and the basal metabolic rate, the metabolic database further configured to store the values for the physiological factors corresponding to the user;
a processor configured to compute the basal metabolic rate and body mass index of the user based on the values of the physiological factors corresponding to the user;
a comparator configured to compare the values of ideal basal metabolic rate and body mass index with the standard values for basal metabolic rate and body mass index, wherein the standard values are selected based on the physiological factors corresponding to the user;
a determining module configured to determine the calorie intake of the user based on the comparison performed by the comparator; and
a first menu generator configured to automatically generate a customized food menu for the user based upon at least the result from the determination module and based on at least calorie content in the items, basal metabolic rate and body mass index of the user.

In accordance with the present disclosure, the system further includes a food repository cooperating with the first menu generator and second menu generator, the food repository configured to store the details corresponding to a plurality of food items, wherein the details include at least names of food items and the nutrient value associated with each of the food items.

In accordance with the present disclosure, the system further includes a calorie database cooperating with the first menu generator and second menu generator, the calorie database configured to store the calorie content value corresponding to each of the food items stored in the food server module.

In accordance with the present disclosure, the user interface is configured to enable the user to specify his/her calorie requirement while requesting for a food menu.

In accordance with the present disclosure, the system further includes:
a receiving module cooperating with the user interface and configured to receive a request for a food menu;
a second determining module configured to determine whether the user has specified a calorie requirement;
a second processing module configured to process the request and process the calorie requirement specified by the user, in the event that the user has specified a calorie requirement; and
a second menu generator cooperating with the food repository and calorie database, the second menu generator configured to generate a food menu based on the calorie requirement specified by the user.

In accordance with the present disclosure, the customized food menu is selected form the group of menus consisting of breakfast menu, lunch menu and dinner menu.

In accordance with the present disclosure, the first menu generator is further configured to generate a customized menu based on at least time at which the user makes the request.

In accordance with the present disclosure, the determining module is configured to determine based on the comparison performed by the comparator, whether the user is having a balanced calorie intake.

In accordance with the present disclosure, the determining module is configured to determine based on the comparison performed by the comparator, whether the user is having an increased/decreased calorie intake.

In accordance with the present disclosure, the first menu generator is configured to generate a customized food menu comprising at least names of food items, calorie content present in each of the food items and nutrients contained in each of the food items.

The present disclosure envisages a computer implemented method for determining, managing and optimizing the calorie intake of a user. The computer implemented method, in accordance with the present disclosure includes the following computer implemented steps:
enabling the user to generate a request for a food menu and receiving from the user values corresponding to a plurality of physiological factors corresponding to the user;
accessing values corresponding to a plurality of physiological factors associated with individuals and deriving standard values for body mass index and basal metabolic rate depending upon the values of the physiological factors;
storing values of the physiological factors and the corresponding standard values for body mass index and the basal metabolic rate and also storing the values for the physiological factors corresponding to the user;
computing the basal metabolic rate and body mass index of the user based on the values of the physiological factors corresponding to the user;
comparing computed values of basal metabolic rate and body mass index with the standard values for basal metabolic rate and body mass index, wherein the standard values are selected based on the physiological factors corresponding to the user; and
determining the calorie intake of the user based on the comparison of values of basal metabolic rate and body mass index and the standard values for basal metabolic rate and body mass index; and
automatically generating a customized food menu for the user based upon at least the result of the comparison and based on at least calorie content in food items, calorie intake of the user; basal metabolic rate and body mass index of the user.

In accordance with the present disclosure, the step of determining the calorie intake of the user further includes: the step of determining whether the user has an increased/decreased calorie intake.

In accordance with the present disclosure, the step of determining the calorie intake of the user further includes the step of determining whether the user has a balanced calorie intake.

In accordance with the present disclosure, the step of enabling the user to generate a request for a food menu further includes the step of enabling the user to specify a calorie requirement along with the request for the food menu.

In accordance with the present disclosure, the step of enabling the user to specify a calorie requirement further includes the following steps:
receiving, from the user, a request for a food menu;
determining whether the user has specified a calorie requirement;
processing the request and processing the calorie requirement specified by the user in the event that the user has specified a calorie requirement; and
generating a food menu based on the calorie requirement specified by the user.

In accordance with the present disclosure, the step of automatically generating a customized menu includes the step of generating customized menus selected form the group consisting of breakfast menu, lunch menu and dinner menu.

In accordance with the present disclosure, the step of automatically generating a customized food menu includes the step of generating a customized food menu based on the calorie intake of the user.

### BRIEF DESCRIPTION OF THE ACCOMPANYING DRAWINGS

The A computer implemented system and method for determining, managing and optimizing calorie intake of a user will now be described with reference to the accompanying, non-limiting drawings, in which:

FIG. 1 illustrates a schematic of a system for determining, managing and optimizing calorie intake of a user; and

FIG. 2 and FIG. 3 include a flowchart illustrating the steps involved in the method for determining, managing and optimizing calorie intake of a user.

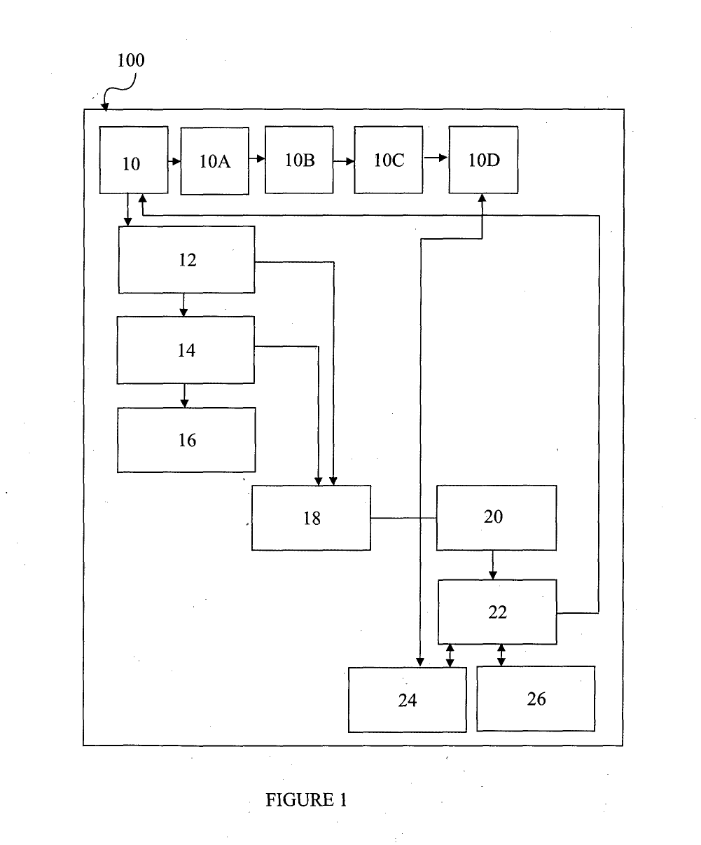

### DETAILED DESCRIPTION OF THE ACCOMPANYING DRAWINGS

The system of the present disclosure will now be described with reference to the embodiments shown in the accompanying drawings. The embodiments do not limit the scope and ambit of the disclosure. The description relates purely to the examples and preferred embodiments of the disclosed method and its suggested applications.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein.

Accordingly, the examples should not be construed as limiting the scope of the embodiments herein.

The health of an individual is often influenced by a plurality of life style related factors including the food habits, number of calories consumed per day, number of calories burnt per day, nature of work and number of hours of sleep. A balanced diet with required amount of nutrients assists people in maintaining good health and also assists people in striking a balance between the number of calories consumed and the number of calories spent/burnt. It is the imbalance between the calorie intake and the calorie expenditure that stimulates various health related issues including high blood sugar level, reduced glucose metabolism, coronary heart disease, kidney failure, low blood sugar level, back pain and breakdown of body tissues to name a few. Therefore, in order to maintain a good health and to ward off most of the diseases it is necessary to have a healthy diet with provides the body with adequate (balanced) number of calories. However, when eating out, it is often difficult if not impossible to determine the amount of nutrients available in the food and the calorie value associated with the food. Moreover, the menu provided in eateries in inflexible and can be altered by the user depending upon the calorie values and the nutritional value associated with the food items. The menu provided in eateries does not contain any information about the calories and nutrients contained in the served food. Hence users visiting eateries would be unable plan their food intake by differentiating between food items rich on calories and food items low on calories.

In order to effectively deal with the aforementioned drawbacks, the present disclosure envisages computer implemented system for determining, managing and optimizing the calorie intake of a user. The system 100 for determining, managing and optimizing the calorie intake of a user will be described with reference to the accompanying drawings which do not restrict the scope and ambit of the present disclosure. The description provided is purely by the way of example and illustration.

In accordance with the present disclosure, the system 100 includes a user interface 10 configured to enable a user to generate a request for a food menu. The user interface 10 can be displayed on any computer enabled device with graphical capabilities. Apart from enabling the user to request for a food menu, the user interface 10 also collects from the user information, typically values corresponding to a plurality of physiological factors associated with the user. The physiological factors corresponding to the user are selected from the group consisting of at least age, height, weight and sex of the user.

The values corresponding to the aforementioned physiological factors, as provided by the user, are transmitted to a processor 12. The processor 12 makes use of these values, i.e., values corresponding to the age, height, weight and sex of the user to calculate the basal metabolic rate (BMR) and body mass index (BMI) of the user. Basal metabolic rate is the number of calories a person burns in a given day if he/she is totally at rest. Body mass index provides a gross estimation of the amount of fat in the human body. The values for the basal metabolic rate and body mass index, computed by the processor 12 are stored along with the corresponding values of the physiological factors, in a database 14. While being stored in the metabolic database 14, the values for basal metabolic rate and body mass index of a user will be mapped with the values of the corresponding physiological features of the user.

In accordance with the present disclosure, the system 100 includes a deriving module denoted by the reference numeral 16. The deriving module 16 has access to the values corresponding to a plurality of physiological factors that are commonly associated with individuals. The values that the deriving module 16 has access to are termed as ‘standard values’ which could have been derived as a result of medical research or as a result of survey or can also be obtained from third party sources. Basically, the deriving module 16 has access to the standard values of basal metabolic rate (BMR) and body mass index (BMI) that a person of a given age, given height, given weight and given sex (male/female) should have given their height, weight, age and sex. The same phenomenon is applied for individuals having different height, different weight, different age and different sex.

The database 14, in accordance with the present disclosure is further configured to store the ‘standard values’ of the basal metabolic rate (BMR) and body mass index (BMI) along with the corresponding ‘standard’ values for age, height, weight and sex of individuals. It is to be noted that the database 14 stored the basal metabolic rate and body mass index of an individual (user) along with the physiological features corresponding to that user, and also the standard values for basal metabolic rate and body mass index corresponding to individuals of various height, various weight, various age and different sex.

The system 100, in accordance with the present disclosure includes a comparator denoted by the reference numeral 18. The comparator 18 is communicably coupled to the database 14. The comparator 18 is configured to compare the values of basal metabolic rate and body mass index of the user (computed by the processor 12) with the standard values of basal metabolic rate and body mass index. The standard values of basal metabolic rate and body mass index are selected form the database 14 such that the age, height, weight and sex of the particular user match with values of the age, sex, height, weight which correspond to the standard values of basal metabolic rate and body mass index.

The result of the aforementioned comparison is passed onto a determining module 20 which is configured to determine the calorie intake of the user. In accordance with the present disclosure the determining module 20 has access to the standard values of the basal metabolic rate and the body mass index, the values of basal metabolic rate and body mass index computed by the processor 12, and also the results of the comparison between the aforementioned set of values, performed by the comparator 18. In accordance with an exemplary embodiment of the present disclosure, if the numerical value of body mass index is in the range ‘24-25’, then it indicates a healthy body weight. If the body mass index is below ‘24’, it indicates underweight and if the body mass index is beyond ‘25’, it indicates a gain in the bodyweight. The comparator 18 makes the comparison between the values of basal metabolic rate and body mass index of the user (computed by the processor 12) with the standard values of basal metabolic rate and body mass index and determines whether the body mass index of the user, amongst other parameters, is above or below or equal to ‘25’. The comparator transmits the results of the comparison to the determining module 20.

The determining module 20 determines that the user is underweight and that his/her calorie intake is low, based on the following procedure. The processor 12 computes the body mass index of the user based on the values of the physiological factors corresponding to the user. The calculated body mass index value is stored in the database 14. The database 14 also stores the standard values corresponding to the body mass index of individuals. The ‘standard value’ of the body mass index is the value that an individual of a particular age, sex, height and weight should have. Prior to selecting the standard value, the user's age, height, weight and height is compared with the values' of height, weight, age and sex stored (pre-stored) in the database 14, and the standard values corresponding to the matched values of height, weight, age and sex stored in the repository will be taken into consideration for the purpose of comparison.

The comparator 18 compares the calculated value of body mass index with the standard value of body mass index, and forwards the results of the comparison to the determining module 20. If the determining module 20 determines that the body mass index of the user is less than what it should have been, given user's age, height, weight and sex (standard value), the determining module 20 concludes that the user is underweight and that the user's calorie intake is lower than what it should have been.

Similarly, if the determining module 20 determines that the body mass index of the user is higher/greater than what it should have been, given user's age, height, weight and sex (standard value), the determining module 20 concludes that the user is overweight and that the user's calorie intake is higher than what it should have been.

The system 100, in accordance with the present disclosure includes a first menu generator denoted by the reference numeral 22. The first menu generator 22 cooperates with a food repository 24 and a calorie database 26. The first menu generator 22 automatically generates a menu customized to meet the requirements of the user, subsequent to the user making a request for a food menu. The first menu generator 22 takes into consideration, the determination of the calorie intake performed by the determining module 20, calorie content present in the food items available for serving, basal metabolic rate of the user and the body mass index of the user. The first menu generator 22 cooperates with the food repository which stores at least the names of the food items available for serving and their respective nutrient values. The food repository also contains information about the ingredients used to prepare each of the food items. The first menu generator 22 further cooperates with the calorie database that also contains the names of food items (the names of food items in the food repository and calorie database are preferably the same) and the number of calories contained in each of the food items.

The first menu generator 22, at first takes into consideration the result of the determination performed by the determining module 20. If the determining module 20 has determined that the calorie intake of the user is on the higher side, then the first menu generator consults the calorie database 26 to identify and retrieve the food items having calorie content matching user's body health condition. Typically, if the user's calorie intake is high, the first menu generator 22 consults the calorie database 26 and retrieves food items having low calorie values. Further, the first menu generator 22 consults the food server 24 to determine the nutrient value contained in the retrieved food items. The first menu generator 22 also consults the database 14 to obtain the values corresponding to basal metabolic rate and body mass index of the user.

Subsequently, the first menu generator 22 generates a customized food menu for the user, wherein the menu is customized based on at least the calorie intake of the user, calorie content of the food items available for serving, basal metabolic rate of the user and body mass index of the user. The menu generated by the first menu generator 22 includes a list of food items along with their calorie values, nutritional values/facts and the ingredients utilized to prepare the food items.

In accordance with the present disclosure, the food menu generated by the first menu generator can be one of a breakfast menu, lunch menu and dinner menu.

In accordance with another aspect of the present disclosure, the user interface 10 is configured to enable the user to specify his/her specific calorie requirement while requesting for a food menu. In case the user specifies his/her calorie requirement, the request from the user is forwarded to a receiving module 10A. The receiving module 10A forwards the request to a second determining module 10B which determines whether the user has specified a calorie requirement. If it is determined that the user has specified a calorie requirement, the request is forwarded to a second processing module 10C which processes the request. The second processing module 10C subsequent to receiving the calorie requirement of the user forwards the request to a second menu generator 10D which in turn consults the calorie database to retrieve food items matching the calorie requirement of the user. Subsequently, the second menu generator 10D generates a food menu having food items that match the calorie values specified by the user. The second menu generator also consults the food repository 24 to determine the nutritional facts/values and the ingredients of the food items deemed to be having the calorie values as specified by the user.

In accordance with the present disclosure, the food menu provided by the second menu generator is one of a breakfast menu, lunch menu and dinner menu. The food menu generated by the first menu generator 22 as well as the second menu generator 10D include at least the names of food items, calorie values of the food items, nutritional facts/values of the food items and ingredients used for preparing the food items. The system 100 in accordance with the present disclosure enables users to optimize his/her calorie requirement by specifying their respective calorie requirement and by providing them with an opportunity to have only those food items that match users' calorie requirement.

IN accordance with the present disclosure, the food menu generated by the first menu generator 22 as well as the second menu generator 10D are communicated to the user through the user interface 10. The first menu generator 22 as well as the second menu generator 10D are also configured to generate a food menu based on the time at which the user makes the request. For example, if the user makes a request for food menu in the night, a menu for dinner would be provided to the user. Similarly, if the user makes a request for food menu in the morning, a menu for breakfast would be provided to the user.

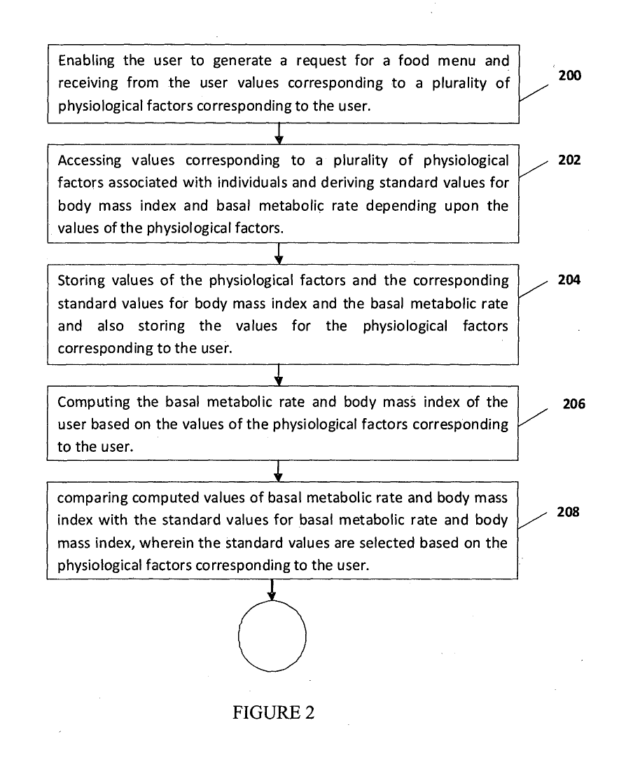

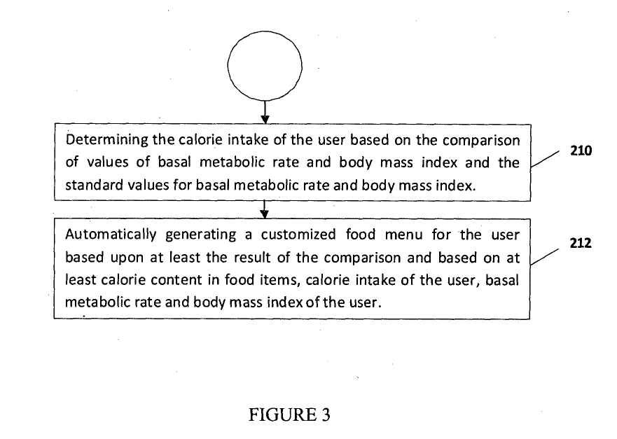

Referring to FIG. 2 and FIG. 3, there is shown a flowchart illustrating the steps involved in the computer implemented method for determining, managing and optimizing calorie intake of a user. The method, in accordance with the present disclosure includes the following computer implemented steps:
enabling the user to generate a request for a food menu and receiving from the user values corresponding to a plurality of physiological factors corresponding to the user 200;
accessing values corresponding to a plurality of physiological factors associated with individuals and deriving standard values for body mass index and basal metabolic rate depending upon the values of the physiological factors 202;
storing values of the physiological factors and the corresponding standard values for body mass index and the basal metabolic rate and also storing the values for the physiological factors corresponding to the user 204;
computing the basal metabolic rate and body mass index of the user based on the values of the physiological factors corresponding to the user 206;
comparing computed values of basal metabolic rate and body mass index with the standard values for basal metabolic rate and body mass index, wherein the standard values are selected based on the physiological factors corresponding to the user 208;
determining the calorie intake of the user based on the comparison of values of basal metabolic rate and body mass index and the standard values for basal metabolic rate and body mass index 210; and
automatically generating a customized food menu for the user based upon at least the result of the comparison and based on at least calorie content in food items, calorie intake of the user, basal metabolic rate and body mass index of the user 212.

In accordance with the present disclosure, the step of determining the calorie intake of the user further includes the step of determining whether the user has an increased/decreased calorie intake.

In accordance with the present disclosure, the step of determining the calorie intake of the user further includes the step of determining whether the user has a balanced calorie intake.

In accordance with the present disclosure, the step of enabling the user to generate a request for a food menu further includes the step of enabling the user to specify a calorie requirement along with the request for the food menu.

In accordance with the present disclosure, the step of enabling the user to specify a calorie requirement further includes the following steps:
receiving, from the user, a request for a food menu;
determining whether the user has specified a calorie requirement;
processing the request and processing the calorie requirement specified by the user in the event that the user has specified a calorie requirement; and
generating a food menu based on the calorie requirement specified by the user.

In accordance with the present disclosure, the step of automatically generating a customized menu includes the step of generating customized menus selected form the group consisting of breakfast menu, lunch menu and dinner menu.

In accordance with the present disclosure, the step of automatically generating a customized food menu includes the step of generating a customized food menu based on the calorie intake of the user.

### TECHNICAL ADVANTAGES

The technical advantages of the system for determining, managing and optimizing calorie intake of a user include the following:
keeping track of the calorie intake of a user;
generating a food menu (menu card) customized according to users' individual needs;
generating a food menu based on the calorie requirement specified by individual users;
assisting a user in maintaining good health by recommending him/her a balanced diet;
assisting users in avoiding health risks; and
assisting users in maintaining/reducing their respective body weight.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the invention, unless there is a statement in the specification specific to the contrary.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A computer implemented system for determining, managing and optimizing calorie intake of a user, said system comprising:
a user interface configured to enable the user to generate a request for a food menu, said user interface further configured to receive from the user values corresponding to a plurality of physiological factors corresponding to the user;
a deriving module having access to the values corresponding to a plurality of physiological factors associated with individuals, said deriving module configured to derive standard values for body mass index and basal metabolic rate depending upon the values of said physiological factors;
a database configured to store values of said physiological factors and the corresponding standard values for body mass index and the basal metabolic rate, said database further configured to store the values for the physiological factors corresponding to the user;
a processor configured to compute the basal metabolic rate and body mass index of the user based on the values of the physiological factors corresponding to the user;
a comparator configured to compare the values of ideal basal metabolic rate and body mass index with the standard values for basal metabolic rate and body mass index, wherein the standard values are selected based on the physiological factors corresponding to the user; and
a determining module configured to determine the calorie intake of the user based on the comparison performed by said comparator; and
a first menu generator configured to automatically generate a customized food menu for the user based upon at least the result from said determination module and based on at least calorie content in the items, basal metabolic rate and body mass index of the user.

2. The system as claimed in claim 1, wherein said system further includes a food repository cooperating with said first menu generator and second menu generator, said food repository configured to store the details corresponding to a plurality of food items, wherein said details include at least names of food items and the nutrient value corresponding to each of said food items.

3. The system as claimed in claim 1, wherein said system further includes a calorie database cooperating with said first menu generator and second menu generator, said calorie database configured to store the calorie content value corresponding to each of the food items stored in said food server module.

4. The system as claimed in claim 1, wherein said user interface is configured to enable the user to specify his/her calorie requirement while requesting for a food menu.

5. The system as claimed in claim 1, wherein said system further includes:
a receiving module cooperating with the user interface and configured to receive a request for a food menu;
a second determining module configured to determine whether the user has specified a calorie requirement;
a second processing module configured to process said request and process the calorie requirement specified by the user, in the event that the user has specified a calorie requirement; and
a second menu generator cooperating with said food repository and calorie database, said second menu generator configured to generate a food menu based on the calorie requirement specified by the user.

6. The system as claimed in claim 1, wherein the customized food menu is selected form the group of menus consisting of breakfast menu, lunch menu and dinner menu.

7. The system as claimed in claim 1, wherein said first menu generator is further configured to generate a customized menu based on at least time at which the user makes the request.

8. The system as claimed in claim 1, wherein said determining module is configured to determine based on the comparison performed by said comparator, whether the user is having a balanced calorie intake.

9. The system as claimed in claim 1, wherein said determining module is configured to determine based on the comparison performed by said comparator, whether the user is having an increased/decreased calorie intake.

10. The system as claimed in claim 1, wherein the first menu generator is configured to generate a customized food menu comprising at least names of food items, calorie content present in each of said food items and nutrients contained in each of said food items.

11. A computer implemented method for determining, managing and optimizing the calorie intake of a user, said computer implemented method comprising the following computer implemented steps:
enabling the user to generate a request for a food menu and receiving from the user values corresponding to a plurality of physiological factors corresponding to the user;
accessing values corresponding to a plurality of physiological factors associated with individuals and deriving standard values for body mass index and basal metabolic rate depending upon the values of said physiological factors;
storing values of said physiological factors and the corresponding standard values for body mass index and the basal metabolic rate and also storing the values for the physiological factors corresponding to the user;
computing the basal metabolic rate and body mass index of the user based on the values of the physiological factors corresponding to the user;
comparing computed values of basal metabolic rate and body mass index with the standard values for basal metabolic rate and body mass index, wherein the standard values are selected based on the physiological factors corresponding to the user;
determining the calorie intake of the user based on the comparison of values of basal metabolic rate and body mass index and the standard values for basal metabolic rate and body mass index; and
automatically generating a customized food menu for the user based upon at least determination of calorie intake of user, calorie content in food items, calorie intake of the user, basal metabolic rate and body mass index of the user.

12. The method as claimed in claim 11, wherein the step of determining the calorie intake of the user further includes the step of determining whether the user has an increased/decreased calorie intake.

13. The method as claimed in claim 11, wherein the step of determining the calorie intake of the user further includes the step of determining whether the user has a balanced calorie intake.

14. The method as claimed in claim 11, wherein the step of enabling the user to generate a request for a food menu further includes the step of enabling the user to specify a calorie requirement along with the request for the food menu.

15. The method as claimed in claim 14, wherein the step of enabling the user to specify a calorie requirement further includes the following steps:
receiving, from the user, a request for a food menu;
determining whether the user has specified a calorie requirement;
processing said request and processing the calorie requirement specified by the user in the event that the user has specified a calorie requirement; and
generating a food menu based on the calorie requirement specified by the user.

16. The method as claimed in claim 11, wherein the step of automatically generating a customized menu includes the step of generating customized menus selected form the group consisting of breakfast menu, lunch menu and dinner menu.

17. The method as claimed in claim 8, wherein the step of automatically generating a customized food menu includes the step of generating a customized food menu based on the calorie intake of the user.
